(19) United States
(12) Patent Application Publication
(10) Pub. No.: US 2025/0000777 A1
(43) Pub. Date: Jan. 2, 2025

(54) MONOPHASE MICELLAR COSMETIC CLEANSING COMPOSITION

(71) Applicant: L'OREAL, Paris (FR)

(72) Inventors: Changlong CHEN, Edison, NJ (US); Christopher PANG, New York, NY (US); Mariko HASEBE, New York, NY (US)

(73) Assignee: L'OREAL, Paris (FR)

(21) Appl. No.: 18/216,832

(22) Filed: Jun. 30, 2023

(51) Int. Cl. A61K 8/73; A61Q 19/10

(52) U.S. Cl. CPC A61K 8/736; A61Q 19/10; A61K 2800/33; A61K 2800/5426; A61K 2800/5428; A61K 2800/596

(57) ABSTRACT

A cosmetic cleansing composition includes a monophasic micellar water that includes at least one amphoteric surfactant present from greater than about 0.05%, by weight, at least one cationic polymer comprising chitosan, polylysine, guar hydroxypropyltrimonium chloride, or combinations thereof present from greater than about 0.01%, by weight, based on the weight of the composition and an aqueous carrier that includes at least water. The cosmetic cleansing composition has a pH in a range from about 4 to about 8, and may be essentially free or free of PEG/POE surfactants. When applied to remove makeup on keratinous tissue the cosmetic cleansing composition demonstrates effective cleansing of longwear foundation and lipstick.

### FIELD OF THE INVENTION

The present disclosure is directed to a skin care composition, in particular, a cosmetic cleansing composition in a micellar formulation.

### BACKGROUND OF THE INVENTION

Monophase micellar water as a gentle makeup remover is popular among consumers as it provides hydration and a refreshing sensation after use. The challenge of monophase micellar water is a relatively low removing efficiency of foundation and mascara, particularly longwear makeup as compared with oil based makeup removers. There is thus a challenge to provide an aesthetically pleasing micellar cleansing composition that provides robust efficacy. Products with alternate cleansing components, including polyethylene glycol surfactants provide an alternative to oil based removers but can prove harsh to the skin.

The inventors have formulated an innovative technology that overcomes the shortcomings of the prior art to improve the performance of micellar water cleanser by incorporating nature-based cationic polymer, for example, but not limited to, chitosan, polylysine, cationic guar, together with amphoteric surfactant. Employing nature based ingredients, the composition lacks ingredients that are harsh or otherwise undesirable or unsustainable and provides comparable or improved efficacy as compared with less natural or harsher compositions. The cosmetic cleansing composition maintains on application a transparent appearance of micellar water with a refreshing sensation after use and provides enhanced removal efficacy and hydration as compared with current product solutions. In some embodiments, the inventive composition is free of polyethylene glycol/polyethylene oxide (PEG/POE) surfactants.

### BRIEF SUMMARY OF THE INVENTION

In various embodiments, provided is a cosmetic cleansing composition that includes a monophasic micellar water comprising:
(a) at least one amphoteric surfactant present in an amount that is greater than about 0.05%, by weight, based on the weight of the composition;
(b) at least one cationic polymer present in an amount that is greater than about 0.01%, by weight, based on the weight of the composition; and
(c) an aqueous carrier comprising at least water.

In some embodiments, the cosmetic cleansing composition is essentially free of PEG/POE surfactants.

In some embodiments, the at least one amphoteric surfactant is present in the cosmetic cleansing composition in a range from about 0.05% to about 10%, by weight, based on the total weight of the cosmetic cleansing composition.

In some embodiments, the at least one amphoteric surfactant is selected from the group consisting of cocamidopropyl hydroxysultaine, coco betaine, cocamidopropyl betaine, sodium lauroamphoacetate, disodium cocoamphodiacetate, and combinations thereof.

In some embodiments, the at least one amphoteric surfactant comprises (i) cocamidopropyl hydroxysultaine present in the cosmetic cleansing composition in a range from about 0.05% to about 10%, (ii) coco-betaine present in the cosmetic cleansing composition in a range from about 0.05% to about 10%, or combinations thereof, all amounts by weight, based on the total weight of the cosmetic cleansing composition.

In some embodiments, each of the at least one cationic polymer is present in an amount that ranges from about 0.01% to about 5% by weight based on the total weight of the cosmetic cleansing composition.

In some embodiments, the at least one cationic polymer is selected from the group consisting of chitosan, polylysine, guar hydroxypropyltrimonium chloride, dextran hydroxypropyltrimonium chloride, and combinations thereof.

In some embodiments, the at least one cationic polymer is selected from (i) chitosan present in the cosmetic cleansing composition in a range from about 0.01% to about 5%, (ii) polylysine present in the cosmetic cleansing composition in a range from about 0.01% to about 5%, (iii) guar hydroxypropyltrimonium chloride present in the cosmetic cleansing composition in a range from about 0.01% to about 5%, or combinations thereof, all amounts by weight, based on the total weight of the cosmetic cleansing composition.

In some embodiments, the at least one cationic polymer is selected from (i) chitosan present in the cosmetic cleansing composition in a range from about 0.05% to about 0.15%, (ii) polylysine present in the cosmetic cleansing composition in a range from about 0.05% to about 0.15%, (iii) guar hydroxypropyltrimonium chloride present in the cosmetic cleansing composition in a range from about 0.05% to about 0.15%, or combinations thereof, all amounts by weight, based on the total weight of the cosmetic cleansing composition.

In some embodiments, the at least one amphoteric surfactant comprises (i) cocamidopropyl hydroxysultaine present in the cosmetic cleansing composition in a range from about 0.5% to about 2.5%, (ii) coco-betaine present in the cosmetic cleansing composition in a range from about 0.1% to about 0.25%, or a combination thereof, and wherein each of the at least one cationic polymer is present in an amount that ranges from about 0.05% to about 2.5% all amounts by weight, based on the total weight of the cosmetic cleansing composition.

In some embodiments, the cosmetic cleansing composition is essentially free of PEG/POE surfactants selected from the group consisting of PEG-20 glyceryl triisostearate, PEG-7 glyceryl cocoate, PEG-20 methylglucoside sesquistearate, PG-5 dioleate, PG-4 diisostearate, PG-10 isostearate, PEG-60 hydrogenated castor oil, PEG-8 stearate, PEG-8 isostearate, PEG-60 hydrogenated castor oil, PEG-150 Laurate, PEG-150 Distearate, PEG-150 pentaerythrityl tetrastearate, PEG-78 Glyceryl Cocoate, PEG-30 Glyceryl Cocoate, Ceteareth-20, Ceteth-10, Ceteth-20, Isoceteth-20, Laureth-4, Laureth-23, Oleth-10, Oleth-20, Steareth-10, Steareth-20, Steareth-100, and Steareth-21, PEG-20 glyceryl laurate, PEG-40 hydrogenated castor oil, poloxamer 184 (polyoxyethylene polyoxypropylene glycol), and combinations thereof.

In some embodiments, the cosmetic cleansing composition also includes one or more co-emulsifier comprising polyglycerol ester selected from the group consisting of polyglyceryl-3 caprylate, polyglyceryl-3 cocoate, polyglyceryl-4 caprate, polyglyceryl-5 laurate, polyglyceryl-6 caprylate, and a combination thereof, and wherein the one or more co-emulsifier is present in the cosmetic cleansing composition in a range from about 0.05% to about 10%, by weight, based on the total weight of the cosmetic cleansing composition.

In some embodiments, the one or more co-emulsifier comprises polyglyceryl-4 caprate present in the cosmetic cleansing composition in a range from about 0.4% to about 1.2%, based on the total weight of the cosmetic cleansing composition.

In some embodiments the cosmetic cleansing composition also includes one or a combination of water-based solvents selected from the group consisting of glycerin, pentylene glycol, and a combination thereof.

In some embodiments, the composition has a pH in the range from about 4.0 to about 8.0.

In some embodiments, the cosmetic cleansing composition also includes at least one of hydroxyacetophenone, caprylyl glycol, sodium chloride, citric acid, tetrasodium glutamate diacetate, or a combination thereof.

In some embodiments, provided is a cosmetic cleansing composition that includes a monophasic micellar water comprising:
(a) at least one amphoteric surfactant comprising (i) cocamidopropyl hydroxysultaine present in the cosmetic cleansing composition in a range from about 0.05% to about 10%, (ii) coco-betaine present in the cosmetic cleansing composition in a range from about 0.05% to about 10%, or combinations thereof;
(b) at least one cationic polymer selected from (i) chitosan present in the cosmetic cleansing composition in a range from about 0.01% to about 5%, (ii) polylysine present in the cosmetic cleansing composition in a range from about 0.01% to about 5%, (ii) guar hydroxypropyltrimonium chloride present in the cosmetic cleansing composition in a range from about 0.01% to about 5%, or combinations thereof;
(c) one or more co-emulsifier is selected from polyglyceryl-4 caprate, glyceryl caprylate, or a combination thereof, and wherein the at least one co-emulsifier is present in the cosmetic cleansing composition in a range from about 0.05% to about 10%;
(d) an aqueous carrier comprising water and one or a combination of water-based solvents selected from the group consisting of glycerin and pentylene glycol; and
(e) at least one of hydroxyacetophenone, caprylyl glycol, sodium chloride, citric acid, tetrasodium glutamate diacetate, or combinations thereof,

all amounts by weight, based on the total weight of the cosmetic cleansing composition, wherein the cosmetic cleansing composition excludes PEG/POE surfactants, and wherein the cosmetic cleansing composition has a pH in the range from about 4.0 to about 8.0.

In some embodiments, the at least one cationic polymer is selected from (i) chitosan present in the cosmetic cleansing composition in a range from about 0.05% to about 0.15%, (ii) polylysine present in the cosmetic cleansing composition in a range from about 0.05% to about 0.15%, (iii) guar hydroxypropyltrimonium chloride present in the cosmetic cleansing composition in a range from about 0.05% to about 0.15%, or combinations thereof, all amounts by weight, based on the total weight of the cosmetic cleansing composition.

In various embodiments, provided is a cosmetic cleansing composition that includes a monophasic micellar water comprising:
(a) at least one amphoteric surfactant present in an amount that is greater than about 0.05%, by weight, based on the weight of the composition;
(b) at least one cationic polymer comprising chitosan, polylysine, guar hydroxypropyltrimonium chloride, or combinations thereof, the at least one cationic polymer present in an amount that is greater than about 0.01%, by weight, based on the weight of the composition; and
(c) an aqueous carrier comprising at least water,

all amounts by weight, based on the total weight of the cosmetic cleansing composition, wherein the cosmetic cleansing composition is essentially free of PEG/POE surfactants, and wherein the cosmetic cleansing composition has a pH of about 5, and wherein the cosmetic cleansing composition when applied to a keratinous tissue demonstrates cleansing of longwear foundation and lipstick.

In some embodiments, the at least one amphoteric surfactant comprises (i) cocamidopropyl hydroxysultaine present in the cosmetic cleansing composition in a range from about 0.05% to about 10%, (ii) coco-betaine present in the cosmetic cleansing composition in a range from about 0.05% to about 10%, or combinations thereof, and wherein the cationic polymer is chitosan present in a range from about 0.01% to about 5%, the cosmetic cleansing composition comprising one or more co-emulsifier, and one or a combination of water-based solvents.

In some embodiments, the one or more co-emulsifier is selected from polyglyceryl-4 caprate, glyceryl caprylate, or a combination thereof present in the cosmetic cleansing composition in a range from about 0.05% to about 10%, and wherein the one or a combination of water-based solvents comprises glycerin and pentylene glycol.

In some embodiments, the cosmetic cleansing composition includes a blend of solvents that include glycerin with one or more of pentylene glycol, caprylyl glycol, butylene glycol, triethyl citrate, propylene glycol or a combination thereof, wherein each solvent is present in a range from about 1% to about 10%, by weight of the cosmetic cleansing composition.

In some embodiments, (a) the at least one cationic polymer is selected from chitosan, guar or a combination thereof, the cosmetic cleansing composition including a blend of water soluble solvents comprising glycerin present at about 1%, and pentylene glycol present at about 3%, or (b) the at least one cationic polymer is polylysine, the cosmetic cleansing composition including a blend of solvents selected from the group consisting of (i) glycerin present at about 1%, pentylene glycol present at about 1.5% and caprylyl glycol present at about 0.5%, (ii) glycerin present at about 1%, butylene glycol present at about 3% and caprylyl glycol present at about 0.5%, (iii) glycerin present at about 1%, triethyl citrate present at about 2.5% and caprylyl glycol present at about 0.5%, (iv) glycerin present at about 1%, triethyl citrate present at about 3% and propylene glycol present at about 3%, and (v) a combination thereof.

According to some embodiments, the composition has a pH in a range from about 4.5 to about 5.5.

According to some embodiments, the composition is essentially free of acrylate type copolymer, anionic surfactants, sulfate and sulfonate based surfactants, glycolipids, or combinations thereof.

It is to be understood that both the foregoing general description and the following detailed description are exemplary and explanatory only and are not restrictive of the invention.

### DETAILED DESCRIPTION OF THE INVENTION

In the various embodiments, the cosmetic cleansing composition is a micellar water essentially lacking an oil phase. In some embodiments, the cosmetic cleansing composition is a skin cleanser, shampoo, or body wash and in some particular embodiments, the composition is a facial cleanser and/or makeup remover.

The cosmetic cleansing composition includes a monophasic micellar water that includes at least one amphoteric surfactant present from greater than about 0.05%, by weight, at least one cationic polymer comprising chitosan, polylysine, guar hydroxypropyltrimonium chloride, or combinations thereof present from greater than about 0.01%, by weight, based on the weight of the composition and an aqueous carrier that includes at least water. The cosmetic cleansing composition is essentially free of PEG/POE surfactants, and has a pH in a range from about 4 to about 8. When applied to a keratinous tissue the cosmetic cleansing composition demonstrates cleansing of longwear foundation and lipstick. The cosmetic cleansing composition in some embodiments is nature based, and formulated to be natural or all natural.

The inventors have demonstrated that incorporating cationic polymer into monophase micellar water, an unexpected and significant improvement in removal efficiency was achieved compared to current benchmark and samples w/o cationic polymer. Without being bound by theory, it is posited that the technology works as positively charged groups in the polycation favors electrostatic attraction with negatively charged groups in surfactants, that is the hydroxyl group in polycation and surfactant also have strong hydrogen bonding with each other. As a result, polycation attracts surfactants to form aggregates/micelles on the cationic polymer chain. It is further posited that the resulting polycation-surfactant associate has a strong complex interaction with makeups via electro attraction, hydrogen bonding, and van der waals force, etc. Thus, it is posited that the polycation serves as delivery vehicle for surfactant to absorb and remove makeup. When the cosmetic cleansing composition adsorbs at makeup surface, it also drags surfactants to the makeup surface where the surfactants are condensed at makeup surface then provide better removal.

Through expert evaluations of makeup removal efficacy, the cosmetic cleansing composition according to the disclosure has demonstrated performance on par with or better than competitor compositions when removing foundation and excels at removing eye and lip makeup.

Ex vivo evaluation of makeup removal demonstrates that the cosmetic cleansing composition performs significantly better than the commercial benchmark, and direct comparisons between compositions with and without the cationic polymer demonstrate a significant effect of the cationic polymer over baseline.

In the various embodiments, the cosmetic cleansing composition is suitable for application to keratinous tissue for cleansing, particularly for removal of makeup. In some embodiments, the cosmetic cleansing composition is essentially free of, free of, or devoid of one or more of oils, silicones, and film formers. In other embodiments, the cosmetic cleansing composition may comprise any one or more of ingredients comprising oils, silicone oils, silicone polymers, and film formers. In some embodiments, the cosmetic cleansing composition is free or essentially free of harsh chemicals and non-nature based ingredients (silicones, alcohol, paraben, fragrance, sulfates).

The cosmetic cleansing composition, can comprise, consist of, or consist essentially of the essential elements and limitations of the invention described herein, as well as any additional or optional ingredients, components, or limitations described herein or otherwise useful.

“Cleanser” or “cleansing composition” as used herein means and refers to any cleansing composition utilized for application to a keratinous tissue for one or more of cleansing the skin, removal of make-up and the like.

“Excluded surfactant” means and refers to excluded surfactants have not been added as a component in the cosmetic cleansing composition. In some particular embodiments, the cosmetic cleansing composition is essentially free, free or devoid of one or more of PEG/PEO surfactants, sulfate/sulfonate based surfactants, and anionic surfactants. Those of skill in the art will appreciate that a surfactant may be present in a composition via its presence in one or more of the formulation components; thus, in some embodiments the cosmetic cleansing composition may be “essentially-free” wherein an excluded surfactant is present at a concentration that does not exceed 5% by weight, and in some instances is present not more than 3% by weight, and in some instances is present not more than 1% by weight, based on the weight of the cosmetic cleansing composition. In some particular embodiments “free” means that the cosmetic cleansing composition is free or devoid specifically of an excluded surfactant. Some specific but non-limiting examples of surfactants that are lacking from the cosmetic cleansing composition include: PEG-20 glyceryl triisostearate, PEG-7 glyceryl cocoate, PEG-20 methylglucoside sesquistearate, PG-5 dioleate, PG-4 diisostearate, PG-10 isostearate, PEG-60 hydrogenated castor oil, PEG-8 stearate, PEG-8 isostearate, PEG-60 hydrogenated castor oil, PEG-150 Laurate, PEG-150 Distearate, PEG-150 pentaerythrityl tetrastearate, PEG-78 Glyceryl Cocoate, PEG-30 Glyceryl Cocoate, Ceteareth-20, Ceteth-10, Ceteth-20, Isoceteth-20, Laureth-4, Laureth-23, Oleth-10, Oleth-20, Steareth-10, Steareth-20, Steareth-100, and Steareth-21, PEG-20 glyceryl laurate, PEG-40 hydrogenated castor oil, poloxamer 184 (polyoxyethylene polyoxypropylene glycol), and combinations thereof.

“Free” and “devoid” and “exclude” or “excluded” each indicates that no reliably measurable excluded material, for example, an excluded volatile silicone oil or excluded surfactant or other excluded material as described herein, is present in the cosmetic cleansing composition. The term “essentially free” means that, while it is preferred that no excluded material is present in the cosmetic cleansing composition, it is possible to have very small amounts of the excluded material in the cosmetic cleansing composition of the invention, provided that these amounts do not materially affect the advantageous properties of the cosmetic cleansing composition. In particular, “essentially free” means that excluded material can be present in the cosmetic cleansing composition at an amount of less than 5%, or less than 4%, or less than 3%, or less than 2%, or less than 1%, or less than 0.1% by weight, based on the total weight of the cosmetic cleansing composition.

“Keratinous substrate” and “keratinous tissue” each includes but is not limited to skin, hair, and nails.

The term “micellar” as used in reference to the inventive cosmetic cleansing composition means that the cosmetic cleansing composition consists of a water phase with suspended micellar aggregates of surfactant and cationic polymer that are easily suspended in the water phase by agitation and which rapidly separate/phase out when the cosmetic cleansing composition is at rest. It should be understood that the cosmetic cleansing composition does not include any significant oil content and thus does not include an oil phase. The cosmetic cleansing composition is shaken or agitated to achieve suspension of the surfactant micelles and cationic polymer prior to application. The pre-application agitation forms a suspension of sufficient uniformity and stability to allow a uniform application to keratinous tissue for the removal of make-up. The cosmetic cleansing composition is light-weight, much like a toner, with a pleasing feel upon application.

“Natural” or “Nature-based” as used herein means and refers to cosmetically acceptable materials and components that are one or more of directly obtained from nature, are obtained from nature with minimal processing, and are derivatives of materials that are obtained from nature. Cleansing composition according to the instant disclosure are in some embodiments up to 99.11% natural, or “Highly natural” or “All Natural” which means that all carbon atoms of intentionally added ingredients are from natural sources per ISO 16128.

“Petrochemical-free” means that excluded petrochemical have not been added as a component. Some specific but non-limiting examples of petrochemicals that are lacking from the cosmetic cleansing composition include benzalkonium chloride, isododecane, isohexadecane and the like. In some embodiments, the cosmetic cleansing composition is not free from petrochemicals, but is formulated to have an amount of petrochemicals that is at or below the amounts found in similar compositions that are commercially available.

“Silicone-free” means that excluded silicones have not been added as a component. In some embodiments, a composition is devoid of silicones. Some specific but non-limiting examples of silicones that are lacking from the cosmetic cleansing composition includes, but is not limited to, silicone polymers, for example selected from dimethicone, cyclopentasiloxane, and other silicone oils, and silicone elastomers.

“Skin” as used herein means and refers to skin materials containing keratin such as facial and body skin, scalp, eyebrows, and lips.

“Stable” and “Stability” as used herein means that the cosmetic cleansing composition does not show any signs of significant changes in one or more of color, odor, pH, or viscosity at room temperature and at temperatures in the range from about 25° C. to about 45° C. In various embodiments, an inventive composition remains stable at temperatures in the range from about 25° C. to about 45° C., over a time period of at least one month, or at least two months, or longer, or any value, range, or sub-range therebetween. In some embodiments, a cleansing composition is stable at 25° C. for at least one month, or at least two months, or longer, or any value, range, or sub-range therebetween. In some embodiments, a cleansing composition is stable at 45° C. for at least one month, or at least two months, or longer, or any value, range, or sub-range therebetween. As described herein, some compositions including anionic polymers in place of the cationic polymer demonstrate cloudiness which is associated with precipitation of one or more ingredients demonstrating instability of the composition.

### Cosmetic Cleansing Composition

### Amphoteric Surfactant

In the various embodiments, the cosmetic cleansing composition comprises at least one amphoteric surfactant (or zwitterionic surfactant). In some embodiments, the cosmetic cleansing composition comprises more than one amphoteric surfactant. In some embodiments, the cosmetic cleansing composition may include or excludes any one or more of nonionic, cationic, or anionic surfactants, and in particular embodiments, excludes PEG/POE surfactants. In some embodiments, the cosmetic cleansing composition includes a combination amphoteric surfactants.

In some embodiments, the at least one amphoteric surfactant may include cocamidopropyl hydroxysultaine, cocamidopropyl betaine, coco betaine, sodium lauroamphoacetate, disodium cocoamphodiacetate, or a combination thereof. In some particular embodiments, the cosmetic cleansing composition includes a combination of surfactants that comprise cocamidopropyl hydroxysultaine, cocamidopropyl betaine, coco betaine, sodium lauroamphoacetate, disodium cocoamphodiacetate, or a combination thereof.

In some embodiments the at least one amphoteric surfactant may be selected from, for example, betaines, alkyl sultaines, alkyl amphoacetates and alkyl amphodiacetates, alkyl amphoproprionates, amphocarboxylates, alkyl betaines, amidoalkyl betaines, amphophosphates, phosphobetaines, pyrophosphobetaines, carboxyalkyl polyamines, amidoalkyl sultaines, salts thereof, or mixtures thereof.

Betaines which can be used in the current compositions include those having the formulas below:

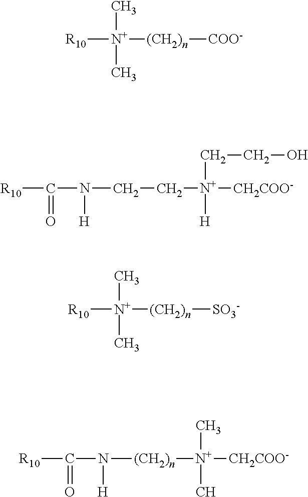

wherein
R¹⁰ is an alkyl group having 8-18 carbon atoms; and
n is an integer from 1 to 3.

Particularly useful betaines include, for example, cocoamidopropyl hydroxysultaine, coco betaine, cocoamidopropyl betaine, lauryl betaine, laurylhydroxy sulfobetaine, lauryldimethyl betaine, behenyl betaine, capryl/capramidopropyl betaine, lauryl hydroxysultaine, stearyl betaine, and mixtures thereof. Typically, the at least one betaine compound is selected from the group consisting of coco betaine, cocoamidopropyl betaine, behenyl betaine, capryl/capramidopropyl betaine, lauryl betaine, and mixtures thereof, and more typically coco betaine.

Hydroxyl sultaines useful in the compositions of the invention include the following

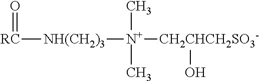

wherein
R is an alkyl group having 8-18 carbon atoms.

More specific examples include, but are not limited to cocamidopropyl hydroxysultaine, lauryl hydroxysultaine, or mixtures thereof.

Useful alkylamphoacetates include those having the formula

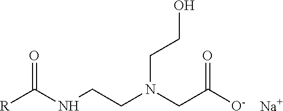

wherein
R is an alkyl group having 8-18 carbon atoms.
useful alkyl amphodiacetates include those having the formula

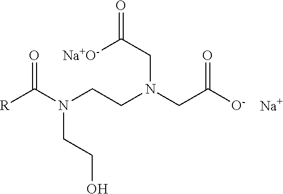

wherein
R is an alkyl group having 8-18 carbon atoms.

### Alkyl Amphopropionates

Exemplary and non-limiting examples of useful alkyl amphopropionates include cocoamphopropionate, caprylamphopropionate, cornamphopropionate, caproamphopropionate, oleoamphopropionate, isostearoamphopropionate, stearoamphopropionate, lauroamphopropionate, salts thereof, or mixtures thereof.

The at least one amphoteric surfactant of the present disclosure may be optionally quaternized secondary or tertiary aliphatic amine derivatives, in which the aliphatic group is a linear or branched chain comprising from 8 to 22 carbon atoms, said amine derivatives containing at least one anionic group, for instance a carboxylate, sulfonate, sulfate, phosphate or phosphonate group.

The at least one amphoteric surfactant may be present in the cosmetic cleansing composition in a range from about 1% to about 5%, based on the weight of the cosmetic cleansing composition.

In various embodiments, each of the at least one amphoteric surfactant may be present in an amount in the range of from about 0.1% to about 20%, and the total amount of surfactant may be present in a range from about 0.1% to about 40% or more. In some embodiments, each of the at least one amphoteric surfactant is present in an amount that is at least 0.1%, or about 1%, to about 20%, or up to 20%, or about 0.1% to about 5%, or to about 3% or to about 8%, or from about 0.05% to about 10%, or from about 5% to about 10%, or from about 7% to about 15%, or from about 10% to about 30%, or about 30% to about 50%, or any value, range, or sub-range therebetween by weight, based on the weight of the cosmetic cleansing composition.

In some embodiments, the at least one amphoteric surfactant includes cocamidopropyl hydroxysultaine present in the cosmetic cleansing composition in a range from about 0.05% to about 10%, or coco-betaine present in the cosmetic cleansing composition in a range from about 0.05% to about 10%, or a combination thereof.

In some embodiments, the at least one amphoteric surfactant includes cocamidopropyl hydroxysultaine present in the cosmetic cleansing composition in a range from about 0.5% to about 2.5%, (ii) coco-betaine present in the cosmetic cleansing composition in a range from about 0.1% to about 0.25%, or a combination thereof.

Thus, in various embodiments, each of the at least one amphoteric surfactant may be present in a composition according to the disclosure from 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 120, 21, 22, 23, 24, 25, 26, 27,28,29, 30, 3, 32, 33, 34, 35, 36, 37, 38, 39, 40, 41, 42, 43, 44, 45, 46, 47, 48, 49 to about 50 percent, including increments and ranges therein and there between.

### Cationic Polymer

In the various embodiments, the cosmetic cleansing composition comprises at least one cationic polymer selected from nature-based polymers that are polysaccharides, and other natural (i.e., plant, animal, or bacterial based), synthetic, or modified cationic nature-based or synthetic polymers.

In some embodiments, the cationic polymer is a nature-based polymer. In general, a cationic nature-based polymer may be selected from cationic forms and cationic derivatives of polysaccharides isolated from algae, polysaccharides produced by microorganisms, and polysaccharides from higher plants, such as homogeneous polysaccharides.

In some embodiments, cationic nature-based polymer selected from polysaccharides may be chosen from polysaccharides that include chitosan, chitin, starches, alginates, celluloses, galactomannans such as guar gums, particularly cationic derivatives thereof, and combinations thereof. In some embodiments, the at least one cationic nature-based polymer selected from polysaccharides may be chosen from chitosan, chitosan derivatives, chitin, starch, starch derivatives, cellulose (for example, but not limited to, ethylcellulose, nitrocellulose, hemicellulose, and hemicellulose derivatives), alginates, including but not limited to, sodium alginate, and combinations thereof.

In some embodiments, the cationic polymer is a nature-based cationic polymer selected from: chitosan, chitosan oligosaccharide, polymeric chitosan having MW in a range from 1 kDa to about 1000 kDa, derivatives of chitosan, derivatives of chitosan having enhanced solubility, cyclodextrin, cationic gelatin, cationic dextran, cationic cellulose, polylysine, polyornithine, histone, collagen, chitosan-cysteine, chitosan-thiobutylamidine, chitosan-thioglycolic acid, or combinations thereof.

Examples of cationic polymers include polysaccharide-based delivery molecules (e.g., chitosan, cyclodextrin, cationic gelatin, cationic dextran, cationic cellulose), cationic peptides and their derivatives (e.g., polylysine, polyornithine), peptide/protein polymers ((e.g., histone, collagen), linear or branched synthetic polymers (e.g., polybrene, polyethyleneimine), natural polymers (e.g., histone, collagen), synthetic dendrimers, cationic thiolated biopolymers (nature-based thiomers or nature-based dendrimers, e.g., chitosan-cysteine, chitosan-thiobutylamidine as well as chitosan-thioglycolic acid).

Examples of nature-based cationic polymers include polysaccharide-based delivery molecules (e.g., chitosan, cyclodextrin, cationic gelatin, cationic dextran, cationic cellulose); cationic peptides and their derivatives (e.g., polylysine, polyornithine), peptide/protein polymers (e.g., histone, collagen); cationic thiolated biopolymers (nature-based thiomers or nature-based dendrimers, e.g., chitosan-cysteine, chitosan-thiobutylamidine, chitosan-thioglycolic acid); or combinations thereof.

In some embodiments, the cosmetic cleansing composition comprises the at least one nature-based cationic chitosan selected from chitosan oligosaccharide, chitosan (or polymeric chitosan having molecular weight (MW) in a range from about 1 kDa to about 1000 kDa), derivatives of chitosan, including derivatives having enhanced solubility, or combinations thereof.

In various embodiments, chitosan has a molecular weight (MW) in a range from about 1 kDa to about 1000 kDa. In some particular embodiments, chitosan has a MW that is “low” and is in the range from about 1 kDa to about 20 kDa, or from about 10 kDa to about 20 kDa, or from about 12 kDa to about 18 kDa. In some embodiments, the chitosan has a Chitosan MW=˜27 kDa

In some embodiments, the at least one nature-based cationic polymer is present from about 0.05% to about 2%, or from about 0.2% to about 0.4%, based on the weight of the cosmetic cleansing composition and comprises chitosan.

In some embodiments, the at least one nature-based cationic polymer is chitosan present in an amount that ranges from about 0.01% to about 5%.

In some embodiments, the at least one nature-based cationic polymer is chitosan present in an amount that ranges from about 0.05% to about 2.5%.

Chitosan is a unique polycation because its charge density depends on the degree of acetylation and pH of the media, and its solubility depends on the degree of acetylation and molecular weight. And while chitosan oligomers are soluble over a wide pH range, from acidic to basic (i.e., physiological pH 7.4), chitosan with increasing molecular weight are soluble only in acidic aqueous media even at high deacetylation degrees. This lack of solubility at neutral and basic pH has hindered the use of chitosan in some applications under neutral physiological conditions (i.e., pH 7.4).

The pH of the cosmetic cleansing composition is in the range from about 4 to about 8, or from about 4 to about 7, or from about 4 to about 6, or from about 4 to about 5, or from about 4.5 to about 6.5, or from about 5 to about 5.5, or from about 5 to about 6, or about 5.

The at least one nature-based cationic polymer is present in the cosmetic cleansing composition according to the disclosure from about 0.01% to about 10%, or from about 0.05% to about 2%, or from about 0.05% to about 1%, or from about 0.1% to about 0.8%, or from about 0.1% to about 0.5%, or from about 0.2% to about 0.4%, or about 0.4%, or about 0.4%, or any suitable combination, sub-combination, range, or sub-range thereof by weight, based on the weight of the cosmetic cleansing composition.

Thus, one or a combination of polymers may be present, by weight, based on the total weight of the cosmetic cleansing composition, from about 0.05, 0.06, 0.07, 0.08, 0.90, 0.1, 0.2, 0.3, 0.4, 0.5, 0.6, 0.7, 0.8, 0.9, 1.0, to about 2 weight percent, including increments and ranges therein and there between.

In some embodiments, the cationic polymer comprises chitosan having MW in a range from 10 kDa to about 20 kDa.

In some particular embodiments, each of the at least one cationic nature-based polymer selected from polysaccharides may be chosen from chitosan and galactomannans such as guar gums, particularly cationic derivatives thereof.

In other embodiments, cationic nature-based polymer selected from polysaccharides may be chosen from one or more of methylcelluloses, hydroxyalkylcelluloses, ethylhydroxyethylcelluloses and carboxymethylcelluloses, mannans, xylans, lignins, arabans, galacturonans, alginate-based compounds, chitin, glucuronoxylans, arabinoxylans, xyloglucans, glucomannans, fructosans such as inulin, pectic acids and pectins, arabinogalactans, agars, glycosaminoglycans, gum arabics, tragacanth gums, ghatti gums, karaya gums, locust bean gums, biopolysaccharide gums of microbial origin such as scleroglucan or xanthan gums, mucopolysaccharides, chondroitin sulfates, and mixtures thereof.

In accordance with the various embodiments, the amount of each of the at least one cationic nature-based polymer present in the cosmetic cleansing compositions can range from about 0.01% to about 10%, or from about 0.01% to about 5%, or from about 0.01% to about 0.5%, or from about 0.1% to about 0.5%, or from about 0.1% to about 0.2%, or from about 0.1% to about 5% or from about 1% to about 3.5% or any suitable combination, sub-combination, range, or sub-range thereof by weight, based on the weight of the cosmetic cleansing composition. One of ordinary skill in the art, however, will appreciate that other ranges are within the scope of the invention.

In the various embodiments, the total amount of cationic nature-based polymer that may be present in the cosmetic cleansing composition can range from about 0.01% to about 20%, or from about 0.05% to about 10%, or from about 0.1% to about 5%, or from about 0.05% to about 1%, or from about 0.01% to about 0.5%, or from about 0.1% to about 0.5%, or from about 0.1% to about 0.2%, or about 0.2% or any suitable combination, sub-combination, range, or sub-range thereof by weight, based on the weight of the cosmetic cleansing composition. One of ordinary skill in the art, however, will appreciate that other ranges are within the scope of the invention.

Thus, any one of the at least cationic nature-based polymer, when present, is present, by weight, based on the total weight of the cosmetic cleansing composition, from about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, to about 10 weight percent, including increments and ranges therein and there between.

In some embodiments, the cationic polymer is not a nature-based polymer and may be selected from synthetic polymers. In some examples, the synthetic polymers are non-silicone based, and in some embodiments may be selected from the group consisting of polyquaterniums, that is polymers having quaternary ammonium centers in the polymer, for example, including polyquaternium-47 (1-Propanaminium,N,N,N-trimethyl-3-[(2-methyl-1-oxo-2-propenyl)amino]-, chloride, polymer with methyl 2-propenoate and 2-propenoic acid). Other non-limiting examples of such polyquaternium compounds may be selected from diallyidimethylammonium chloride/acrylic acid copolymers sold under the names MERQUAT 280 POLYMER or MERQUAT 280NP POLYMER or MERQUAT 281 POLYMER or MERQUAT 295 POLYMER, by the company Nalco (Lubrizol) (INCI name: Polyquaternium-22); the copolymer of methacrylamidopropyltrimonium chloride, of acrylic acid and or methyl acrylate, sold under the name MERQUAT 2001 POLYMER OR MERQUAT 2001N POLYMER by the company Nalco (Lubrizol) (INCI name: Polyquaternium-47); the acrylamide/dimethyldiallylammonium chloride/acrylic acid terpolymer sold under the name MERQUAT 3330DRY POLYMER or MERQUAT 3330PR POLYMER or MERQUAT 3331PR POLYMER or MERQUAT 3940 POLYMER or MERQUAT PLUS 3330 POLYMER OR MERQUAT PLUS 3331 POLYMER by the company Nalco (Lubrizol) (INCI name:: Polyquaternium-39); an ampholytic terpolymer consisting of methacrylamidopropyl trimethyl ammonium chloride (MAPTAC), acrylamide and acrylic acid, sold under the name MERQUAT 2003PR POLYMER by the company Nalco (Lubrizol) (INCI name: : Polyquaternium-53); Polyquaternium-30, Polyquaternium-35, Polyquaternium-45, Polyquaternium-50, Polyquaternium-63; Polyquaternium-74; Polyquaternium-54; Polyquaternium-57; Polyquaternium-76; Polyquaternium-86; Polyquaternium-89; Polyquaternium-95; Polyquaternium-98, Polyquaternium-104; Polyquaternium-111; Polyquaternium-112, and mixtures thereof. In some embodiments, the cationic polymer is a synthetic polymer selected from cationic acrylic polymers, for example, an amorphous functional acrylic polymer grafted onto a polyethylene backbone such as SYNTRAN™ 5330, which is a quaternary modified olefin grafted technology. More generally, such synthetic polymers selected from, but are not limited to, cationic polymers comprising polyacrylates such as those identified in the International Cosmetic Ingredient Dictionary and Handbook (9 th ed. 2002) such as, for example, polyacrylate-1, polyacrylate-2, polyacrylate-3, polyacrylate-4, polyacrylate-16, polyacrylate-17, polyacrylate-18, polyacrylate-19, polyacrylate-21, and mixtures thereof. Such (co)polymers, or similar (co)polymers, can be combined individually or with other (co)polymers in such a way to form suitable bimodal agents having both cationic and anionic functionalities.

In the various embodiments, the amount of cationic polymer is not a nature-based polymer and is selected from synthetic cationic polymers that may be present in the cosmetic cleansing composition can range from about 0.01% to about 20%, or from about 0.05% to about 10%, or from about 0.1% to about 5%, or from about 0.5% to about 2%, or about 1%, or from about 0.05% to about 1%, or from about 0.01% to about 0.5%, or from about 0.1% to about 0.5%, or from about 0.1% to about 0.2% or any suitable combination, sub-combination, range, or sub-range thereof by weight, based on the weight of the cosmetic cleansing composition.

Thus, any one of the at least synthetic cationic polymer, when present, is present, by weight, based on the total weight of the cosmetic cleansing composition, from about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, to about 10 weight percent, including increments and ranges therein and there between.

In the various embodiments, the amount of each of the at least one cationic polymer which may be either nature-based or synthetic cationic polymer, alone or in combination with another cationic polymer, present in the cosmetic cleansing compositions can range from about 0.01% to about 10%, or from about 0.01% to about 5%, or from about 0.01% to about 0.5%, or from about 0.1% to about 0.5%, or from about 0.1% to about 0.2%, or from about 0.1% to about 5% or from about 1% to about 3.5%, or from about 0.5% to about 2%, or about 1%, or any suitable combination, sub-combination, range, or sub-range thereof by weight, based on the weight of the cosmetic cleansing composition. One of ordinary skill in the art, however, will appreciate that other ranges are within the scope of the invention.

### Surfactant/Co-Emulsifier (Co-Emulsifier)

In some embodiments the cosmetic cleansing composition may include one or more surfactant of co-emulsifier (herein co-emulsifier).

In some embodiments, suitable co-emulsifier that may be used according to the disclosure may be chosen from Polyglyceryl-4 Caprate (PG-4 caprate), Glyceryl caprylate, isopropyl myristate, isopropyl palmitate, ethylhexyl palmitate, methyl palmitate, 2-ethylhexyl palmitate, hexyl laurate, ethylhexyl laurate, isopropyl laurate, ethylhexyl oleate, ethylhexyl isononanoate, isononyl isononanoate, myristyl myristate, 2-ethylhexyl caprate/caprylate (or octyl caprate/caprylate), isostearyl neopentanoate, cetearyl ethylhexanoate, isopropyl isostearate, diisopropyl sebacate, coco caprylate/caprate, diisopropyl adipate, and combinations thereof esters of lactic acid and of fatty alcohols comprising 12 or 13 carbon atoms, or combinations thereof.

In some embodiments, the one or more co-emulsifier is present from about 1% to about 8% by weight, based on the weight of the cosmetic cleansing composition. In some embodiments, the total amount of co-emulsifier does not exceed a total of about 10% by weight of the cosmetic cleansing composition.

In some embodiments, each of the one or more co-emulsifier may be present from about 0.05% to about 10%, or from about 0.1% to about 15% by weight of the cosmetic cleansing composition, or from about 1% to about 10% by weight, or from about 1% to about 8%, or from about 0.05% to about 2%, or about 1% or any suitable combination, sub-combination, range, or sub-range thereof by weight, based on the weight of the cosmetic cleansing composition. When present, the total amount of surfactant/co-emulsifier present in the cosmetic cleansing composition may be in a range of from about 0.1% to about 15% by weight, or from about 1% to about 8% by weight, or any suitable combination, sub-combination, range, or sub-range thereof by weight, based on the weight of the cosmetic cleansing composition. One of ordinary skill in the art, however, will appreciate that other ranges are within the scope of the invention.

Thus, each of the one or more co-emulsifier in the cosmetic cleansing composition may be present by weight, based on the total weight of the cosmetic cleansing composition, from about 0.5, 0.6, 0.7, 0.8, 0.9, 0.1, 0.2, 0.3, 0.4, 0.5, 0.6, 0.7, 0.8, 0.9, 1, 2, 3, 4, 5, 6, 7, 8, 9, 10, 11, 12, 13, 14 to about 15 percent, including increments and ranges therein and there between.

### Water

In the various embodiments, the cosmetic cleansing composition includes water.

In various embodiments, the cosmetic cleansing composition comprises from about 5% to about 90% water, and in some embodiments from about 20% to about 85% water, and in some embodiments from about 50% to about 85% water, and in some embodiments from about 60% to about 80% water, and in some embodiments from about 65% to about 75%, water, including increments and all ranges and subranges therein and there between, by weight, based on the total weight of the cosmetic cleansing composition. One of ordinary skill in the art, however, will appreciate that other ranges are within the scope of the invention. Thus, water is present, by weight, based on the total weight of the cosmetic cleansing composition, from about 5, 10, 15, 20, 25, 30, 35, 40, 45, 50, 55, 60, 65, 66, 67, 68, 69, 70, 71, 72, 73, 74, 75, 80, 85, to about 90 weight percent, including increments and all ranges and subranges therein and there between.

The water used in the cosmetic cleansing composition may be sterile demineralized water and/or a floral water such as rose water, cornflower water, chamomile water or lime water, and/or a natural thermal or mineral water such as, for example: water from Vittel, water from the Vichy basin, water from Uriage, water from La Roche Posay, water from La Bourboule, water from Enghien-les-Bains, water from Saint Gervais-les-Bains, water from Neris-les-Bains, water from Allevar-les-Bains, water from Digne, water from Maizieres, water from Neyrac-les-Bains, water from Lons-le-Saunier, water from Eaux Bonnes, water from Rochefort, water from Saint Christau, water from Les Fumades, water from Tercis-les-Bains or water from Avene. The water phase may also comprise reconstituted thermal water, that is to say a water comprising trace elements such as zinc, copper, magnesium, etc., reconstituting the characteristics of a thermal water.

Generally, the pH can be adjusted to the desired value by addition of a base or an acid (organic or inorganic). In view of the inclusion of hydroxy acids in the cosmetic cleansing composition hereof, the pH is typically adjusted using a base, which in some examples is sodium hydroxide, or using an acid, which in some examples is citric acid.

### Water-Soluble Solvents

In some embodiments, the cosmetic cleansing composition may include at least one water-soluble solvent. The term “water-soluble solvent” is interchangeable with the term “water-miscible solvent” and means a compound that is liquid at 25° C. and at atmospheric pressure (760 mmHg), and it has a solubility of at least 50% in water under these conditions. In some cases, the water-soluble solvent has a solubility of at least 60%, 70%, 80%, or 90% in water under these conditions.

In some embodiments, the cosmetic cleansing composition includes a blend of solvents that include glycerin with one or more of pentylene glycol, caprylyl glycol, butylene glycol, triethyl and propylene glycol. In such embodiments, each of the solvents is present from about 1% to about 10%, by weight of the composition.

In some embodiments, the cosmetic cleansing composition includes a blend of solvents that includes at least glycerin with additional solvents including pentylene glycol and caprylyl glycol, butylene glycol and caprylyl glycol, triethyl citrate and caprylyl glycol, or triethyl citrate and propylene glycol.

In some particular embodiments, the cosmetic cleansing composition includes a blend of water soluble solvents selected from the group consisting of glycerin, pentylene glycol and caprylyl glycol, glycerin, butylene glycol and caprylyl glycol, glycerin, triethyl citrate and caprylyl glycol, glycerin, triethyl citrate and propylene glycol, or combinations thereof.

In some particular embodiments, the cosmetic cleansing composition which includes one of chitosan or guar or a combination thereof as the cationic polymer includes a blend of water soluble solvents selected from the group consisting of glycerin present at about 1%, and pentylene glycol present at about 3%.

In some particular embodiments, the cosmetic cleansing composition which includes polylysine as the cationic polymer includes a blend of water soluble solvents selected from the group consisting of glycerin present at about 1%, pentylene glycol present at about 1.5% and caprylyl glycol present at about 0.5%, glycerin present at about 1%, butylene glycol present at about 3% and caprylyl glycol present at about 0.5%, glycerin present at about 1%, triethyl citrate present at about 2.5% and caprylyl glycol present at about 0.5%, glycerin present at about 1%, triethyl citrate present at about 3% and propylene glycol present at about 3%, or combinations thereof.

Non-limiting examples of water-soluble solvents include, for example, glycerin, alcohols (for example, C1-C30, C1-C15, C1-C10, or C1-C4 alcohols), polyols, glycols, and combinations thereof.

As examples of organic solvents, non-limiting mentions can be made of monoalcohols and polyols such as ethyl alcohol, isopropyl alcohol, propyl alcohol, benzyl alcohol, and phenylethyl alcohol, or glycols or glycol ethers such as, for example, monomethyl, monoethyl and monobutyl ethers of ethylene glycol, propylene glycol or ethers thereof such as, for example, monomethyl ether of propylene glycol, butylene glycol, hexylene glycol, dipropylene glycol as well as alkyl ethers of diethylene glycol, for example monoethyl ether or monobutyl ether of diethylene glycol. Other suitable examples of organic solvents are ethylene glycol, propylene glycol, butylene glycol, hexylene glycol, propane diol, and glycerin. The organic solvents can be volatile or non-volatile compounds.

Further non-limiting examples of water-soluble solvents include alkanols (polyhydric alcohols such as glycols and polyols) such as glycerin, 1,2,6-hexanetriol, trimethylolpropane, ethylene glycol, propylene glycol, diethylene glycol, butylene glycol, hexylene glycol, triethylene glycol, tetraethylene glycol, pentaethylene glycol, dipropylene glycol, 1,3-butanediol, 2,3-butanediol, 1,4-butanediol, 3-methyl-1,3-butanediol, 1,5-pentanediol, tetraethylene glycol, 1,6-hexanediol, 2-methyl-2,4-pentanediol, polyethylene glycol, 1,2,4-butanetriol, 1,2,6-hexanetriol, 2-butene-1,4-diol, 2-ethyl-1,3-hexanediol, 2-methyl-2,4-pentanediol, (caprylyl glycol), 1,2-hexanediol, 1,2-pentanediol, and 4-methyl-1,2-pentanediol; alkyl alcohols having 1 to 4 carbon atoms such as ethanol, methanol, butanol, propanol, and isopropanol; glycol ethers such as ethylene glycol monomethyl ether, ethylene glycol monoethyl ether, ethylene glycol monobutyl ether, ethylene glycol monomethyl ether acetate, diethylene glycol monomethyl ether, diethylene glycol monoethyl ether, diethylene glycol mono-n-propyl ether, ethylene glycol mono-iso-propyl ether, diethylene glycol mono-iso-propyl ether, ethylene glycol mono-n-butyl ether, ethylene glycol mono-t-butyl ether, diethylene glycol mono-t-butyl ether, 1-methyl-1-methoxybutanol, propylene glycol monomethyl ether, propylene glycol monoethyl ether, propylene glycol mono-t-butyl ether, propylene glycol mono-n-propyl ether, propylene glycol mono-iso-propyl ether, dipropylene glycol monomethyl ether, dipropylene glycol monoethyl ether, dipropylene glycol mono-n-propyl ether, and dipropylene glycol mono-iso-propyl ether; 2-pyrrolidone, N-methyl-2-pyrrolidone, 1,3-dimethyl-2-imidazolidinone, formamide, acetamide, dimethyl sulfoxide, sorbit, sorbitan, acetine, diacetine, triacetine, sulfolane, and combinations thereof.

In some cases, a water-soluble solvent may be selected from the group consisting of pentylene glycol, glycerin, and combinations thereof.

In the various embodiments total amount of the at least one water-soluble solvent, when present, may vary, is from about 0.5% to about 25%, or from about 0.5% to about 20%, or from about 1% to about 20%, or from about 1% to about 10%, or from about 2% to about 5% or any suitable combination, sub-combination, range, or sub-range thereof by weight, based on the weight of the cosmetic cleansing composition. One of ordinary skill in the art, however, will appreciate that other ranges are within the scope of the invention.

Thus, any one water-soluble solvent, when present, is present, by weight, based on the total weight of the cosmetic cleansing composition, from about 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24 to about 25 weight percent, including increments and ranges therein and there between.

### Preservatives

In some embodiments, one or more preservatives and/or antimicrobials may be present in the cosmetic cleansing composition.

Any preservative commonly used in cosmetic cleansing compositions is an acceptable preservative for the cosmetic cleansing compositions herein, such as phenoxyethanol, members from the paraben family such as the methyl, ethyl, propyl, butyl or isobutyl parabens, 4-hydroxy benzoic acid, benzoic acid, sorbic acid, dehydroacetic acid, triclosan, benzyl alcohol, chlorophenesin, or salicylic acid, for example. At more concentrated amounts of suitable solvents for optional additives, in particular, suitable solvents for antimicrobials and preservatives, members from the paraben family may be used as a preservative.

In some embodiments, the one or more preservatives, when present, may be selected from caprylyl glycol, Myrtrimonium bromide, phenoxyethanol, hydroxyacetophenone, ethylhexyl glycerin, chlorphenesin, cetrimonium chloride, hexyl glycerin, octylglycerin, benzylglycerin, 3-heptoyl-2,2-propandiol, and 1,2-hexandiol; polyaminopropyl biguanide, also known as polyhexamethylene biguanide, or PHMB, and combinations thereof. In some embodiments, the cosmetic cleansing composition is free or essentially free of one or more of caprylyl glycol, phenoxyethanol, hexyl glycerin, ethylhexylglycerin, octylglycerin, benzylglycerin, 3-heptoyl-2,2-propandiol, and 1,2-hexandiol; polyaminopropyl biguanide, also known as polyhexamethylene biguanide, or PHMB.

In some embodiments, the one more preservatives includes caprylyl glycol.

In some embodiments, the preservative may comprise one or more of preservatives selected from the group consisting of organic acids, parabens, formaldehyde donors, phenol derivatives, quaternary ammoniums, alcohols, isothiazolinones, and combinations thereof. Preservatives having antibacterial activity are optionally present in the cosmetic cleansing compositions of the present invention. Examples of organic acid preservatives include, but are not limited to, sodium benzoate, potassium sorbate, benzoic acid and dehydroacetic acid, sorbic acid, and combinations thereof. A preferred organic acid preservative system includes a mixture of sodium benzoate and potassium sorbate. Examples of paraben preservatives include, but are not limited to, alkyl para-hydroxybenzoates, wherein the alkyl radical has from 1, 2, 3, 4, 5 or 6 carbon atoms and for example, from 1 to 4 carbon atoms e.g., methyl para-hydroxybenzoate (methylparaben), ethyl para-hydroxybenzoate (ethylparaben), propyl para-hydroxybenzoate (propylparaben), butyl para-hydroxybenzoate (butylparaben) and isobutyl para-hydroxybenzoate (isobutylparaben). Examples of formaldehyde donor preservatives include, but are not limited to, 1,3-Dimethylol-5,5-dimethylhydantoin (DMDM hydantoin), imidazolidinyl urea, gluteraldehyde, and combinations thereof. Examples of quaternary ammonium preservatives include, but are not limited to, benzalkonium chloride, methene ammonium chloride, benzethonium chloride, and combinations thereof. Examples of alcohol preservatives include, but are not limited to, ethanol, benzyl alcohol, dichlorobenzyl alcohol, phenoxyethanol, and combinations thereof. Examples of isothiazolone preservatives include, but are not limited to, methylchloroisothiazolinone, methylisothiazolinone, and combinations thereof.

In some particular embodiments, appropriate preservatives include, but are not limited to, phenoxyethanol, Tetrasodium EDTA, Zinc Gluconate, Hydroxyacetophenone, Caprylyl glycol, Chlorphenesin, or combinations thereof.

In some embodiments, the preservative includes one or more preservatives, the one or combination present at a concentration, by weight of about 0.001% to about 5%, or alternatively about 0.05% to about 2.5% or alternatively about 0.1% to about 2.0%, or from about 0.02% to about 1% by weight, or from about 0.03% to about 0.5%, or any suitable combination, sub-combination, range, or sub-range thereof by weight, based upon weight of the cosmetic cleansing composition. One of ordinary skill in the art, however, will appreciate that other ranges are within the scope of the invention.

Thus, any one of or a combination of preservatives, when present, may be present, by weight, based on the total weight of the cosmetic cleansing composition, is from about 0.001, 0.002, 0.003,0.004, 0.005, 0.006, 0.007, 0.008, 0.009, 0.01, 0.02, 0.03,0.04, 0.05, 0.06, 0.07, 0.08, 0.09, 1.0, 1.1, 1.2, 1.3, 1.4, 1.5, 1.6, 1.7, 1.8, 1.9, 2, 3, 4, to about 5 weight percent, including increments and ranges therein and there between.

### Optional Additives

In some embodiments, the cosmetic cleansing composition includes one or more of other optional ingredients selected from the group consisting of skin care actives, humectants, conditioning agents, thickeners, viscosity adjusters, cooling agents, fillers, antimicrobials, preservatives, pH adjusters, chelating agents, and combinations thereof.

In some embodiments, a cleansing composition includes at least one additive used in the cosmetics field which does not affect the properties of the cosmetic cleansing composition according to the invention, such as, fragrances, pH adjusters (citric acid, sodium chloride, lactic acid); neutralizing or pH-adjusting agents (e.g., triethylamine (TEA) and sodium hydroxide, Trisodium ethylenediamine disuccinate, and combinations thereof), other cosmetically acceptable additives, such as but not limited to, pearlescent agents, silica, and coloring materials; essential oils; fruit extracts, for example, Pyrus Malus (Apple) Fruit Extract, and Aloe Barbadensis Leaf Juice Powder; actives (for example, hydroxyacetophenone); vitamins (for example, vitamin A, beta carotene, tocopherol/vitamin E, panthenol, retinol, resveratrol, vitamin C, niacinamide, derivatives thereof, and combinations thereof); hyaluronic acid in the form of hydrolyzed hyaluronic acid or sodium hyaluronate; coloring materials/pigments; essential oils; antioxidants (phenolic compounds, such as chalcones, flavones, flavanones, flavanols, flavonols, dihydroflavonols, isoflavonoids, neoflavonoids, catechins, anthocyanidins, tannins, lignans, aurones, stilbenoids, curcuminoids, alkylphenols, betacyanins, capsacinoids, hydroxybenzoketones, methoxyphenols, naphthoquinones, and phenolic terpenes, resveratrol, curcumin, pinoresinol, ferulic acid, hydroxytyrosol, cinnamic acid, caffeic acid, p-coumaric acid, baicalin (Scutellaria Baicalensis root extract), pine bark extract (Pinus Pinaster bark/bud extract), ellagic acid); vitamins and vitamin derivatives, such as calcium pantothenate, tocopherol and ascorbic acid; hydroxy acids; citric acid, sodium citrate, sodium chloride; neutralizing, chelating or pH-adjusting agents (for example, triethylamine (TEA), trisodium ethylenediamine disuccinate, EDTA, and sodium hydroxide); powders, fragrances, dyes, pigments; organic or mineral UV filters; conditioning agents such as C12-15 Alkyl lactate and C12-15 Alcohols; Thickeners, for example Ammonium polyacryloyldimethyl taurate, Propylene glycol and PEG-55 propylene glycol oleate; viscosity adjusters, for example, Sodium chloride and Hexylene Glycol; cooling agents such as menthol; fillers, for example Kaolin; or any combination thereof. Although the optional additives are given as examples, it will be appreciated that other optional components compatible with cosmetic applications known in the art may be used.

In the various embodiments, the amount of one or more additives, alone or in combination, present in the cosmetic cleansing composition can be present in a range from about 0.0001% to about 20%, and in some embodiments, from about 0.005% to about 0.01%, and in some embodiments, from about 0.01% to about 0.1%, and in some embodiments, from about 0.15% to about 5%, and in some embodiments, from about 0.40% to about 4%, and in some embodiments, from about 0.5% to about 2.5%, and in some embodiments, from about 0.1% to about 0.5% and in some embodiments, from about 1% to about 2%, or any suitable combination, sub-combination, range, or sub-range thereof by weight, based on the total weight of the cosmetic cleansing composition. One of ordinary skill in the art, however, will appreciate that other ranges are within the scope of the invention.

Thus, any one or a combination of additives may be present, by weight, based on the total weight of the cosmetic cleansing composition, each one or the combination present from about 0.0001, 0.0005, 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.10, 0.20, 0.30, 0.40, 0.50, 0.60, 0.70, 0.80, 0.90, 1.0, 2, 3, 4, 5, 6, 7, 8, 9, 10, 11, 12, 13, 14, 15, 16, 17, 18, 19 to about 20 weight percent, including increments and ranges therein and there between.

In some embodiments, the cosmetic cleansing composition may include one or more oils. In some embodiments, the cosmetic cleansing composition is essentially free, free of or devoid of oils.

In some examples, the oils may be chosen from hydrocarbon-based oils. For example, the hydrocarbon-based oil may be a saturated hydrocarbon, an unsaturated hydrocarbon, lipids, triglycerides, a natural oil, and/or a synthetic oil. In some embodiments, the cosmetic cleansing composition may include a synthetic oil selected from the group consisting of hydrogenated polyisobutene and hydrogenated polydecene. A hydrocarbon-based oil may be a non-volatile hydrocarbon-based, such as:
(i) hydrocarbon-based oils of plant origin, such as glyceride triesters, which are generally triesters of fatty acids and of glycerol, the fatty acids of which can have varied chain lengths from C4 to C24, it being possible for these chains to be saturated or unsaturated and linear or branched; these oils are in particular wheat germ oil, sunflower oil, grape seed oil, sesame oil, corn oil, apricot oil, castor oil, shea oil, avocado oil, olive oil, soybean oil, sweet almond oil, palm oil, rapeseed oil, cottonseed oil, hazelnut oil, macadamia oil, jojoba oil, alfalfa oil, poppy oil, pumpkin seed oil, marrow oil, blackcurrant oil, evening primrose oil, millet oil, barley oil, quinoa oil, rye oil, safflower oil, candlenut oil, passionflower oil, musk rose oil, and essential oils, such as Helianthus Annuus Seed Oil, Lavandula Angustifolia (lavender) Oil, Mentha Piperita Oil, Rosmarinus Officinalis (rosemary) Leaf Oil Pelargonium Graveolens flower oil, Citrus Aurantium Dulcis (orange) peel oil, Menthe Viridis (spearmint) leaf oil, Citrus Aurantifolia (lime) oil, Melaleuca Alternifolia (tea tree) leaf oil, Citrus Grandis (grapefruit) peel oil, Citrus Medica Limonum (lemon) peel oil, rose flower oil, eucalyptus globulus leaf oil, and combinations thereof.
(ii) synthetic ethers containing from 10 to 40 carbon atoms;
(iii) linear or branched hydrocarbons of mineral or synthetic origin, such as petroleum jelly, polydecenes, hydrogenated polyisobutene such as Parleam, and 40 squalane;
(iv) synthetic esters, for instance oils of formula RCOOR' in which R represents a linear or branched fatty acid residue containing from 1 to 40 carbon atoms and R′ represents a hydrocarbon-based chain that is especially branched, containing from 1 to 40 carbon atoms on condition that R+R′ is ÿ 10, for instance Purcellin oil (cetearyl octanoate), isopropyl myristate, isopropyl palmitate, C12-C15 alkyl benzoate, such as the product sold under the trade name Finsolv TN™ or Witconol TN™ by Witco or Tegosoft TN™ by Evonik Goldschmidt, 2-ethylphenyl benzoate, such as the commercial product sold under the name X-Tend 226 by ISP, isopropyl lanolate, hexyl laurate, diisopropyl adipate, isononyl isononanoate, oleyl erucate, 2-ethylhexyl palmitate, isostearyl isostearate, diisopropyl sebacate, such as the product sold under the name of “Dub Dis” by Stearinerie Dubois, octanoates, decanoates or ricinoleates of alcohols or polyalcohols, such as propylene glycol dioctanoate; hydroxylated esters, such as isostearyl lactate or diisostearyl malate; and pentaerythritol esters; citrates or tartrates, such as di(linear C12-C13 alkyl) tartrates, such as those sold under the name Cosmacol ETI™ by Enichem Augusta Industriale, and also di(linear C14-C15 alkyl) tartrates, such as those sold under the name Cosmacol ETL™ by the same company; or acetates;
(v) fatty alcohols that are liquid at room temperature, containing a branched and/or unsaturated carbon-based chain containing from 12 to 26 carbon atoms, for instance octyldodecanol, isostearyl alcohol, oleyl alcohol, 2-hexyldecanol, 2-butyloctanol or 2-undecylpentadecanol;
(vi) higher fatty acids, such as oleic acid, linoleic acid or linolenic acid;
(vii) carbonates, such as dicaprylyl carbonate, such as the product sold under the name Cetiol CC™ by Cognis;
(viii) fatty amides, such as isopropyl N-lauroyl sarcosinate, such as the product sold under the trade name Eldew SL 205™ from Ajinomoto; and
(ix) essential oils selected from the group consisting of sunflower oil, sesame oil, peppermint oil, macadamia nut oil, tea tree oil, evening primrose oil, sage oil, rosemary oil, coriander oil, thyme oil, pimento berries oil, rose oil, anise oil, balsam oil, bergamot oil, rosewood oil, cedar oil, chamomile oil, sage oil, clary sage oil, clove oil, cypress oil, eucalyptus oil, fennel oil, sea fennel oil, frankincense oil, geranium oil, ginger oil, grapefruit oil, jasmine oil, juniper oil, lavender oil, lemon oil, lemongrass oil, lime oil, mandarin oil, marjoram oil, myrrh oil, neroli oil, orange oil, patchouli oil, pepper oil, black pepper oil, petitgrain oil, pine oil, rose otto oil, rosemary oil, sandalwood oil, spearmint oil, spikenard oil, vetiver oil, wintergreen oil, and ylang ylang.

In some embodiments the cosmetic cleansing composition may include at least one branched or linear liquid alkane with carbon chain length of C11 to C20. In various embodiments, liquid alkanes may be selected from those with a carbon chain length of from C11 to C20. The liquid alkanes may be selected from those with a carbon chain length of from C11 to C20, or from C15 to C19, or one of C11, C12, C13, C14, C15, C16, C17, C18 to C19. In some particular embodiments, suitable liquid alkanes that may be used according to the disclosure include hydrocarbon-based oils containing from 8 to 16 carbon atoms, and especially branched C8-C16 alkanes such as C8-C16 isoalkanes. In some exemplary embodiments, such liquid alkanes may be chosen from isoparaffins, for instance isododecane (also known as 2,2,4,4,6-pentamethylheptane), isodecane, and isohexadecane.

The amount of each of the at least one branched or linear liquid alkane may be present in the cosmetic cleansing composition in a range of from about 1% to about 35% by weight, or from about 15% to about 30% by weight, or from about 10% to about 20% or any suitable combination, sub-combination, range, or sub-range thereof by weight, based on the weight of the cosmetic cleansing composition. One of ordinary skill in the art, however, will appreciate that other ranges are within the scope of the invention.

Thus, each of the at least one branched or linear liquid alkane in the cosmetic cleansing composition may be present by weight, based on the total weight of the cosmetic cleansing composition, from about 1, 2, 3, 4, 5, 6, 7, 8, 9, 10, 11, 12, 13, 14, 15, 16, 17, 128, 19, 20, 21, 22, 23, 24, 25, 26, 27, 28, 29, 30, 31, 32, 33, 34, to about 35 percent, including increments and ranges therein and there between.

Although the optional additives are given as examples, it will be appreciated that other optional components compatible with cosmetic applications known in the art may be used that are suitable. It will be appreciated by a skilled artisan that any optional additives are present only to the extent and in amounts that do not materially adversely affect the basic and novel characteristic(s) of the claimed disclosure. Thus, in some embodiments that include optional additives, such optional additives will not materially adversely affect the cosmetic cleansing composition.

More generally, it will be appreciated by a skilled artisan that any solvents, humectants, preservatives or other additives are present only to the extent and in amounts that do not materially adversely affect the basic and novel characteristic(s) of the claimed invention.

### EXAMPLES

The following examples are intended to further illustrate the present disclosure. They are not intended to limit the disclosure in any way. Unless otherwise indicated, all parts are by weight.

### Raw Materials

TABLE 1
Materials used include:

| | % Active (if <~100%); |
|---|---|
| | components/solvents in |
| Raw Material | addition to active |
| Chitosan | 85%; (includes beta Glucan at 15%) |
| Polylysine | 25% |
| Cocamidopropyl Hydroxysultaine | 50% |
| Coco Betaine | 30% |
| Polyglyceryl-4 Caprate | 90% |

### Example 1: Compositions

Exemplary embodiments of cosmetic cleansing composition are set forth in Table 1, below.

TABLE 2
Inventive Compositions

| Ingredient | INV 1 | INV 2 | INV 3 | INV 4 | INV 5 | INV 6 | INV 7 | INV 8 | INV 9 |
|---|---|---|---|---|---|---|---|---|---|
| CAPRYLYL GLYCOL | | | | | | | | 0.50 | |
| GLYCERYL CAPRYLATE | 0.50 | 0.50 | 0.50 | | 0.50 | | | | |
| POLYEPSILON-LYSINE | | | | | 0.10 | 0.10 | 0.10 | 0.10 | 0.4 |
| TETRASODIUM GLUTAMATE | | 0.12 | | | | | | | |
| DIACETATE | | | | | | | | | |
| PENTYLENE GLYCOL | 3.00 | 3.00 | 3.00 | 3.00 | 3.00 | 3.00 | 3.00 | 1.50 | 1.0 |
| GLYCERIN | 1.00 | 1.00 | 1.00 | 1.00 | 1.00 | 1.00 | 1.00 | 1.00 | 1.00 |
| CITRIC ACID | 0.05 | 0.05 | 0.005 | 0.005 | 0.05 | 0.05 | 0.05 | 0.05 | 0.05 |
| GUAR | | 0.10 | 0.10 | 0.10 | | | | | |
| HYDROXYPROPYLTRIMONIUM | | | | | | | | | |
| CHLORIDE | | | | | | | | | |
| COCAMIDOPROPYL | 0.40 | 0.40 | 0.60 | 0.60 | 0.60 | 0.60 | | | 1.2 |
| HYDROXYSULTAINE | | | | | | | | | |
| COCO-BETAINE | | | | | | | 0.201 | 0.201 | |
| POLYGLYCERYL-4 CAPRATE | 0.999 | 0.999 | 0.396 | 0.396 | 0.396 | 0.396 | 0.801 | 0.801 | 0.44 |
| SODIUM CHLORIDE | | | | | | | 0.04 | 0.04 | |
| WATER/QS | ~94 | ~94 | ~94 | ~94 | ~94 | ~94 | ~94 | ~95 | ~96 |
| CHITOSAN | 0.085 | | | | | | | | |
| BETA-GLUCAN | 0.015 | | | | | | | | |
| HYDROXYACETOPHENONE | | | | 0.30 | | 0.30 | 0.30 | 0.30 | 0.3 |

Table 3 provides examples of inventive and comparative compositions. A, B, C, D are inventive micellar water formulations with chitosan; A1, B1, C1, D1 are comparative micellar water without chitosan as baselines to illustrate the benefit of the cationic polymer, chitosan.

TABLE 3
Inventive and Comparative compositions

| | Inventive | | | | Comparative | | | |
|---|---|---|---|---|---|---|---|---|
| INCI (US/UE) | With Chitosan | | | | Without Chitosan | | | |
| Formulation Identifier | A | B | C | D | A1 | B1 | C1 | D1 |
| WATER/AQUA | 92.71 | 92.71 | 93.24 | 93.24 | 92.81 | 92.81 | 93.34 | 93.34 |
| CHITOSAN | 0.1 | 0.1 | 0.1 | 0.1 | | | | |
| COCAMIDOPROPYL | 2 | 2 | 0.8 | 0.8 | 2 | 2 | 0.8 | 0.8 |
| HYDROXYSULTAINE | | | | | | | | |
| PG-4 CAPRATE | 0.44 | 0.44 | 1.11 | 1.11 | 0.44 | 0.44 | 1.11 | 1.11 |
| GLYCERYL CAPRYLATE | 0.5 | | 0.5 | | 0.5 | | 0.5 | |
| HYDROXYACETOPHENONE | | 0.5 | | 0.5 | | 0.5 | | 0.5 |
| GLYCERIN | 1 | 1 | 1 | 1 | 1 | 1 | 1 | 1 |
| PENTYLENE GLYCOL | 3 | 3 | 3 | 3 | 3 | 3 | 3 | 3 |
| pH | 5.0 | 5.0 | 5.0 | 5.0 | 5.0 | 5.0 | 5.0 | 5.0 |

TABLE 4
Inventive Compositions with Polylysine & Varied Solvents

| | INV 8 | | | |
|---|---|---|---|---|
| | (from table 2) | VAR 1 | VAR 2 | VAR3 |
| | pentylene glycol + | butylene glycol + | triethyl citrate + | triethyl citrate + |
| Ingredient | caprylyl glycol | caprylyl glycol | caprylyl glycol | propylene glycol |
| COCO-BETAINE | ~0.2 | ~0.2 | ~0.2 | ~0.2 |
| WATER | ~95 | ~93 | ~94 | ~91 |
| GLYCERIN | 1 | 1 | 1 | 1 |
| POLYGLYCERYL-4 | ~0.8 | ~0.8 | ~0.8 | ~0.8 |
| CAPRATE | | | | |
| HYDROXYACETOPHENONE | 0.3 | 0.3 | 0.3 | 0.3 |
| PENTYLENE | 1.5 | | | |
| GLYCOL | | | | |
| butylene glycol | | 3 | | |
| Triethyl citrate | | | 2.5 | 3 |
| PROPYLENE | | | | 3 |
| GLYCOL | | | | |
| CAPRYLYL | 0.5 | 0.5 | 0.5 | |
| GLYCOL | | | | |
| CITRIC ACID | 0.05 | 0.05 | 0.05 | 0.05 |
| POLYEPSILON- | ~0.1 | ~0.1 | ~0.1 | ~0.1 |
| LYSINE | | | | |

### Benchmark Composition #1 Comparative (COMP 1) (Commercial Micellar Water With PEG Surfactant, No Cationic Polymer)

Water, hexylene glycol, glycerin, disodium cocoamphodiacetate, poloxamer 184 (PEG surfactant), preservative/chelator.

### Benchmark Composition #2 Comparative (COMP 2)

MILK MICELLAR WATER

Aloe barbadensis leaf juice, hexylene glycol, aqua (water), glycerin, poloxamer 184, Agave tequilana leaf extract, Cannabis sativa (hemp) seed extract, caffeine, niacinamide, disodium cocoamphodiacetate, citric acid, 1,2-hexanediol, caprylyl glycol, glycolipids, polylysine, sodium hydroxide, sodium hyaluronate, phenoxyethanol, sodium benzoate, potassium sorbate.

### Inventive Compositions

### Example 2. In Vitro Long Wear Foundation Removability Efficiency Removal Efficiency

Foundation: Maybelline's Super Stay Longwear Liquid Foundation

Referring to Table 3, the indicated compositions were tested and compared with the benchmark composition #1. The results are shown below in Table 5.

TABLE 5
Percent removal

| Composition | A | B | C | D | A1 | B1 | C1 | D1 | COMP 1 |
|---|---|---|---|---|---|---|---|---|---|
| Removal % | 43 | 35 | 47 | 43 | 28 | 22 | 26 | 22 | 19 |

The results show that formulations lacking chitosan (A1-D1) demonstrate removal efficiency around 22%-28%. In contrast, inventive compositions with chitosan (A-D) demonstrate removal efficiency around 35%-47%. The performance of the inventive compositions is significantly better than the commercial benchmark COMP 1.

### Example 3. In Vitro Long Wear Foundation Removability Efficiency; Residual After Cleansing

Compositions were evaluated for removability of foundation makeup from artificial skin protein substrate based on color change.

Method of determination of color change: The color difference associated with removal of makeup, ΔE (delta-E; color difference in a rectangular coordinate system), value was measured by Datacolor 600.

Clean reference: completely clean substrate, without makeup applied.

Black reference: substrate with makeup applied, but before removal.

Sample after removal: substrate with makeup applied, and after removal performed.

The equation below was used to calculate the removal efficacy.

$$Removability = \frac{{De}_{sample after removal} - {De}_{black reference}}{{De}_{clean reference} - {De}_{black reference}}$$

Referring to Table 3, the indicated compositions were tested and compared with the benchmark #1 COMP 1. The results are shown below in Table 6.

TABLE 6
retained foundation on skin (delta E)

| Formulations with chitosan | A (23.5) | B (25.8) | C (20.4) | D (20.7) | |
|---|---|---|---|---|---|
| (delta E of residual) | | | | | |
| Formulations without chitosan | A1 (29.4) | B1 (31.0) | C1 (30.0) | D1 (30.6) | COMP 1 |
| (delta E of residual) | | | | | (34.6) |

Less residual foundation was measured on the substrate cleaned with chitosan containing compositions as compared to compositions that did not include chitosan, demonstrating the improvement of makeup removal efficiency by chitosan.

### Example 4. In Vivo Long Wear Makeup Removability Efficiency

Compositions were evaluated for removability of lipstick and mascara.

Maybelline Matt ink super stay lipstick and Maybelline Big shot waterproof mascara were applied to skin of clinical subjects for In vivo removability test on tester arms against long wear lipstick and waterproof mascara.

After lipstick and mascara dried, 0.4 gram of each evaluated composition formulation was applied to remove the makeup.

As compared to Benchmark #1 COMP 1, Inventive composition from Table 3, inventive C (with chitosan), picked up more makeup on cotton rounds and left less residual on tester arms.

### Example 5. In Vivo Long Wear Makeup Removability Efficiency With Varied Cationic Polymer

Compositions were evaluated for removability of lipstick and mascara.

Maybelline Matt ink super stay lipstick and Maybelline Big shot waterproof mascara were applied to skin of clinical subjects for In vivo removability test on tester arms against long wear lipstick and waterproof mascara.

After lipstick and mascara dried, 0.4 gram of each evaluated composition formulation was applied to remove the makeup.

Three different cationic polymers were evaluated in a base composition with hydroxysultaine with and without a co-emulsifier, polyglyceryl-4 caprate. The tested compositions included 0.1% chitosan, 0.1% polylysine, and 0.1% guar.

All three showed improved makeup removability as compared with Benchmark #1 COMP 1, and the addition of the co-emulsifier did not appreciably affect the removability. It is posited that at acidic conditions, protonated chitosan is positively charged, and via electrostatic interaction, sulfonate group of hydroxysultaine amphoteric surfactant in the inventive cosmetic cleansing composition may be strongly bonded to activated amino group to form surfactant aggregates/micelles.

### Example 6. Evaluation of Cationic Polymers Vs Other Bio Polymers

Compositions were evaluated for removability of foundation in the presence of different bio polymers selected from cationic, anionic, and nonionic.

Super stay foundation was applied to skin of clinical subjects for In vivo removability.

Each of the following polymers were tested in both 6/4 hydroxysultaine to polyglyceryl-4 caprate and 2/8 hydroxysultaine to polyglyceryl-4 caprate bases: chitosan, polylysine, and guar (all cationic), xanthan and carrageenan (polyanionic), and sclerotium and pullulan (nonionic).

The results show that the anionic polymers xanthan and carrageenan demonstrate some enhancement in in vitro foundation removal, comparable to the polycations, likely due to counterion attraction or hydrogen bonding between anionic polymer and sultaine/PG-4. The nonionic polymer demonstrated significantly less effectiveness than polycation and polyanion polymers. In vivo mascara test results show that polyanions carrageenan are comparable or slightly better than the Benchmark #1 COMP 1, but do not perform as well as the polycations, which may be due to the polyanion-surfactant complex having a negative charge, not favorable to attraction with makeup/skin surface. Further, formulations with these anionic polymer are either phase separation or very cloudy.

### Example 7. Effect of Polylysine as Cationic Polymer Vs PEG Surfactant w Polylysine Added Polymers

Compositions were evaluated for removability of foundation makeup from artificial skin protein substrate. Referring to Table 1, inventive 8 was evaluated vs benchmark 1 COMP 1 (which includes PEG surfactant) with added polylysine, and benchmark 2 COMP 2. The results are shown below.

TABLE 7
Removal efficiency

| | Benchmark #1 COMP 1 | |
|---|---|---|
| | (which includes PEG | |
| | surfactant) with added | Benchmark #2 |
| Inventive 8 | polylysine | COMP 2 |
| 45.7% | 19.7% | 35.2% |

The results demonstrate that the inventive composition proved more effective in removing makeup, and addition of polycation (polylysine) did not improve the effectiveness of the Benchmark that includes PEG surfactant.

### Example 8. Effect of Polylysine as Cationic Polymer

Compositions were evaluated for removability of foundation makeup from the skin substrate. Referring to Table 1, inventive 8 was evaluated vs the same formulation without polylysine.

The results demonstrate that the inventive composition proved more effective removing makeup, as evidenced by a removal efficiency of 45.7% for the inventive vs 31.3% for the comparative (inventive minus polylysine). Additional cotton pad was needed to remove mascara and lipstick using the comparative. Similar results were obtained with removal of mascara and lipstick, where the inventive composition outperformed each of the benchmark composition #1 and the comparative (inventive minus polylysine).

### Example 9. Effect of Surfactant Ratio, Amount of Polylysine, pH in Range from 4 to 6 and Osmotic Pressure

The effects of varying amphoteric surfactant to co-emulsifier ratios demonstrated that there is no significant impact on performance, however, higher amounts of a co-emulsifier polyglyceryl-4 caprate poorly affected stability at low temperatures. Variation of pH demonstrated that lower pH (around 4) shows better removability attributed likely to greater protonation of polylysine, though generally pH>=4.5 is recommended for eye safety. Variation of polylysine level from 0.1% to 0.5% did not show significant difference in performance. Evaluation of osmotic pressure of compositions with polylysine and guar demonstrate they are comparable to benchmark #1, which together with other commercial makeup removers demonstrate osmotic pressure that is greater than that of tears (at 304 mOsm/L). It is understood in the art that Osmotic pressure between 100-500 mOsm/L is considered safe. Table 8, below, provides osmotic pressure data for inventive and comparative compositions.

TABLE 8
osmotic pressure of various compositions

| | Benchmark | | | | |
|---|---|---|---|---|---|
| COMPOSITION | #1 COMP 1 | INV 9 | INV 3 | INV 6 | INV 5 |
| OSMOTIC | 451 | 276 | 484 | 480 | 468 |
| PRESSURE | | | | | |
| (mOsm/L) | | | | | |

### Example 9. Effect of Solvents (Combination of Two Solvents)

Referring now to the compositions in Table 4, solvents were varied in a base formulation corresponding to INV 8 (VAR 1, VAR 2, VAR 3). The results are shown in Table 9, below.

TABLE 9
Effect of Solvents

| | INV 8 | | | |
|---|---|---|---|---|
| | (from table 2) | VAR 1 | VAR 2 | VAR3 |
| Ingredient | pentylene glycol + | butylene glycol + | triethyl citrate + | triethyl citrate + |
| Solvent mix | caprylyl glycol | caprylyl glycol | caprylyl glycol | propylene glycol |
| Removability | 51.5% | 49.0% | 62.3% | 49.6% |

Referring to Table 9, the results show that all of the tested inventive compositions including INV 8 and the variants VAR 1-VAR 3 accomplish removability of makeup from artificial skin protein substrate of at least about 50%. The data further show that a combination of triethyl citrate and caprylyl glycol in the composition also including glycerin demonstrated enhanced performance, suggesting that inclusion of triethyl citrate and caprylyl glycol can further enhance the efficacy of removability of makeup in embodiments of the inventive composition according to the disclosure.

While the disclosure has been described with reference to described embodiments, it will be understood by those skilled in the art that various changes may be made, and equivalents may be substituted for elements thereof without departing from the scope of the disclosure. In addition, many modifications may be made to adapt a particular situation or material to the teachings of the disclosure without departing from the essential scope thereof. Therefore, it is intended that the disclosure is not limited to the particular embodiment disclosed as the best mode contemplated for carrying out this disclosure, but that the disclosure will include all embodiments falling within the scope of the appended claims.

The articles “a” and “an,” as used herein, mean one or more when applied to any feature in embodiments of the present disclosure described in the specification and claims. The use of “a” and “an” does not limit the meaning to a single feature unless such a limit is specifically stated. The article “the” preceding singular or plural nouns or noun phrases denotes a particular specified feature or particular specified features and may have a singular or plural connotation depending upon the context in which it is used. The adjective “any” means one, some, or all indiscriminately of whatever quantity.

“At least one” or “one or more” as used herein, means that there may be one, two, three or more and thus includes individual components as well as mixtures/combinations.

The transitional terms “comprising,” “consisting essentially of” and “consisting of”, when used in the appended claims, in original and amended form, define the claim scope with respect to what unrecited additional claim elements or steps, if any, are excluded from the scope of the claim(s). The term “comprising” is intended to be inclusive or open-ended and does not exclude any additional, unrecited element, method, step or material. The term “consisting of” excludes any element, step or material other than those specified in the claim and, in the latter instance, impurities ordinarily associated with the specified material(s). The term “consisting essentially of” limits the scope of a claim to the specified elements, steps or material(s) and those that do not materially affect the basic and novel characteristic(s) of the claimed disclosure. All materials and methods described herein that embody the present disclosure can, in alternate embodiments, be more specifically defined by any of the transitional terms “comprising,” “consisting essentially of,” and “consisting of.”

The terms “free” and “devoid” indicates that no reliably measurable excluded material is present in the cosmetic cleansing composition, typically 0% by weight, based on the total weight of the cosmetic cleansing composition. The term “essentially free” means that, while it prefers that no excluded material is present in the cosmetic cleansing composition, it is possible to have very small amounts of the excluded material in the cosmetic cleansing composition of the invention, provided that these amounts do not materially affect the advantageous properties of the cosmetic cleansing composition. In particular, “essentially free” means that excluded material can be present in the cosmetic cleansing composition at an amount of less than about 0.1% by weight, based on the total weight of the cosmetic cleansing composition.

Other than in the operating examples, or where otherwise indicated, all numbers expressing quantities of ingredients and/or reaction conditions are to be understood as being modified in all instances by the term “about,” meaning within 10% of the indicated number (e.g., “about 10%” means 9%-11% and “about 2%” means 1.8%-2.2%).

All percentages and ratios are calculated by weight unless otherwise indicated. All percentages are calculated based on the total composition unless otherwise indicated. Generally, unless otherwise expressly stated herein, “weight” or “amount” as used herein with respect to the percent amount of an ingredient refers to the amount of the raw material comprising the ingredient, wherein the raw material may be described herein to comprise less than and up to 100% activity of the ingredient. Therefore, weight percent of an active in a composition is represented as the amount of raw material containing the active that is used and may or may not reflect the final percentage of the active, wherein the final percentage of the active is dependent on the weight percent of active in the raw material.

All ranges and amounts given herein are intended to include subranges and amounts using any disclosed point as an end point. Thus, a range of “1% to 10%, such as 2% to 8%, such as 3% to 5%,” is intended to encompass ranges of “1% to 8%,” “1% to 5%,” “2% to 10%,” and so on. Similarly, a range given of “about 1% to 10%” is intended to have the term “about” modifying both the 1% and the 10% endpoints. Further, it is understood that when an amount of a component is given, it is intended to signify the amount of the active material unless otherwise specifically stated. As used herein, all ranges provided are meant to include every specific range within, and combination of subranges between, the given ranges. Thus, a range from 1-5, includes specifically 1, 2, 3, 4 and 5, as well as subranges such as and 2-5, 3-5, 2-3, 2-4, 1-4, etc. As used herein a range of ratios is meant to include every specific ratio within, and combination of subranges between the given ranges.

Notwithstanding that the numerical ranges and parameters setting forth the broad scope of the disclosure are approximations, unless otherwise indicated the numerical values set forth in the specific examples are reported as precisely as possible. Any numerical value, however, inherently contains certain errors necessarily resulting from the standard deviation found in their respective testing measurements. The example that follows serves to illustrate embodiments of the present disclosure without, however, being limiting in nature.

All publications and patent applications cited in this specification are herein incorporated by reference, and for any and all purposes, as if each individual publication or patent application were specifically and individually indicated to be incorporated by reference. In the event of an inconsistency between the present disclosure and any publications or patent application incorporated herein by reference, the present disclosure controls.

1. A cosmetic cleansing composition comprising:
a monophasic micellar water comprising:
(a) at least one amphoteric surfactant present in an amount that is greater than about 0.05%, by weight, based on the weight of the composition;
(b) at least one cationic polymer present in an amount that is greater than about 0.01%, by weight, based on the weight of the composition; and
(c) an aqueous carrier comprising at least water,
wherein the cosmetic cleansing composition is essentially free of PEG/POE surfactants.

2. The cosmetic cleansing composition according to claim 1, wherein the at least one amphoteric surfactant is present in the cosmetic cleansing composition in a range from about 0.05% to about 10%, by weight, based on the total weight of the cosmetic cleansing composition, and is selected from the group consisting of cocamidopropyl hydroxysultaine, cocamidopropyl betaine, coco betaine, sodium lauroamphoacetate, disodium cocoamphodiacetate, and combinations thereof.

3. The cosmetic cleansing composition according to claim 1, wherein the at least one amphoteric surfactant comprises (i) cocamidopropyl hydroxysultaine present in the cosmetic cleansing composition in a range from about 0.05% to about 10%, (ii) cocobetaine present in the cosmetic cleansing composition in a range from about 0.05% to about 10%, or combinations thereof, all amounts by weight, based on the total weight of the cosmetic cleansing composition.

4. The cosmetic cleansing composition according to claim 1, wherein each of the at least one cationic polymer is present in an amount that ranges from about 0.01% to about 5% by weight based on the total weight of the cosmetic cleansing composition, and is selected from the group consisting of chitosan, polylysine, guar hydroxypropyltrimonium chloride, dextran hydroxypropyltrimonium chloride, and combinations thereof.

5. The cosmetic cleansing composition according to claim 1, wherein the at least one cationic polymer is selected from the group consisting of (i) chitosan present in the cosmetic cleansing composition in a range from about 0.01% to about 5%, (ii) polylysine present in the cosmetic cleansing composition in a range from about 0.01% to about 5%, or (iii) guar hydroxypropyltrimonium chloride present in the cosmetic cleansing composition in a range from about 0.01% to about 5%, or combinations thereof, all amounts by weight, based on the total weight of the cosmetic cleansing composition.

6. The cosmetic cleansing composition according to claim 1, wherein the at least one cationic polymer is selected from the group consisting of (i) chitosan present in the cosmetic cleansing composition in a range from about 0.05% to about 0.15%, (ii) polylysine present in the cosmetic cleansing composition in a range from about 0.05% to about 0.15%, or (iii) guar hydroxypropyltrimonium chloride present in the cosmetic cleansing composition in a range from about 0.05% to about 0.15%, or combinations thereof, all amounts by weight, based on the total weight of the cosmetic cleansing composition.

7. The cosmetic cleansing composition according to claim 1, wherein the at least one amphoteric surfactant comprises (i) cocamidopropyl hydroxysultaine present in the cosmetic cleansing composition in a range from about 0.5% to about 2.5%, and (ii) coco-betaine present in the cosmetic cleansing composition in a range from about 0.1% to about 0.25%, or a combination thereof, and wherein each of the at least one cationic polymer is present in an amount that ranges from about 0.05% to about 2.5% all amounts by weight, based on the total weight of the cosmetic cleansing composition.

8. The cosmetic cleansing composition according to claim 1, including a blend of solvents that include glycerin and one or more additional solvents selected from the group consisting of pentylene glycol, caprylyl glycol, butylene glycol, triethyl citrate, propylene glycol, and a combination thereof, wherein each solvent is present in a range from about 1% to about 10%, by weight of the cosmetic cleansing composition.

9. The cosmetic cleansing composition according to claim 1, wherein (a) the at least one cationic polymer is selected from chitosan, guar or a combination thereof, the cosmetic cleansing composition including a blend of water soluble solvents comprising glycerin present at about 1%, and pentylene glycol present at about 3%, or (b) the at least one cationic polymer is polylysine, the cosmetic cleansing composition including a blend of solvents selected from the group consisting of (i) glycerin present at about 1%, pentylene glycol present at about 1.5% and caprylyl glycol present at about 0.5%, (ii) glycerin present at about 1%, butylene glycol present at about 3% and caprylyl glycol present at about 0.5%, (iii) glycerin present at about 1%, triethyl citrate present at about 2.5% and caprylyl glycol present at about 0.5%, (iv) glycerin present at about 1%, triethyl citrate present at about 3% and propylene glycol present at about 3%, and (v) a combination thereof.

10. The cosmetic cleansing composition according to claim 1, wherein the cosmetic cleansing composition is essentially free of PEG/POE surfactants selected from the group consisting of PEG-20 glyceryl triisostearate, PEG-7 glyceryl cocoate, PEG-20 methylglucoside sesquistearate, PG-5 dioleate, PG-4 diisostearate, PG-10 isostearate, PEG-60 hydrogenated castor oil, PEG-8 stearate, PEG-8 isostearate, PEG-60 hydrogenated castor oil, PEG-150 Laurate, PEG-150 Distearate, PEG-150 pentaerythrityl tetrastearate, PEG-78 Glyceryl Cocoate, PEG-30 Glyceryl Cocoate, Ceteareth-20, Ceteth-10, Ceteth-20, Isoceteth-20, Laureth-4, Laureth-23, Oleth-10, Oleth-20, Steareth-10, Steareth-20, Steareth-100, and Steareth-21, PEG-20 glyceryl laurate, PEG-40 hydrogenated castor oil, poloxamer 184 (polyoxyethylene polyoxypropylene glycol), and combinations thereof.

11. The cosmetic cleansing composition according to claim 1, comprising one or more co-emulsifier comprising a polyglycerol ester selected from the group consisting of polyglyceryl-3 caprylate, polyglyceryl-3 cocoate, polyglyceryl-4 caprate, polyglyceryl-5 laurate, polyglyceryl-6 caprylate, and a combination thereof, and wherein the one or more co-emulsifier is present in the cosmetic cleansing composition in a range from about 0.05% to about 10%, by weight, based on the total weight of the cosmetic cleansing composition.

12. The cosmetic cleansing composition according to claim 11, wherein the one or more co-emulsifier comprises polyglyceryl-4 caprate present in the cosmetic cleansing composition in a range from about 0.4% to about 1.2%, by weight, based on the total weight of the cosmetic cleansing composition.

13. The cosmetic cleansing composition according to claim 1, comprising one or a combination of water-based solvents selected from the group consisting of glycerin, pentylene glycol, er and a combination thereof.

14. The cosmetic cleansing composition according to claim 1, wherein the composition has a pH in the range from about 4.0 to about 8.0.

15. The cosmetic cleansing composition according to claim 1, comprising at least one of hydroxyacetophenone, caprylyl glycol, sodium chloride, citric acid, tetrasodium glutamate diacetate, or combinations thereof.

16. A cosmetic cleansing composition comprising:
a monophasic micellar water comprising:
(a) at least one amphoteric surfactant comprising (i) cocamidopropyl hydroxysultaine present in the cosmetic cleansing composition in a range from about 0.05% to about 10%, (ii) coco-betaine present in the cosmetic cleansing composition in a range from about 0.05% to about 10%, or combinations thereof;
(b) at least one cationic polymer selected from (i) chitosan present in the cosmetic cleansing composition in a range from about 0.01% to about 5%, (ii) polylysine present in the cosmetic cleansing composition in a range from about 0.01% to about 5%, (ii) guar hydroxypropyltrimonium chloride present in the cosmetic cleansing composition in a range from about 0.01% to about 5%, or combinations thereof;
(c) one or more co-emulsifier is selected from polyglyceryl-4 caprate, glyceryl caprylate, or a combination thereof, and wherein the at least one co-emulsifier is present in the cosmetic cleansing composition in a range from about 0.05% to about 10%;
(d) an aqueous carrier comprising water and one or a combination of water-based solvents selected from the group consisting of glycerin, pentylene glycol and a combination thereof; and
(e) at least one of hydroxyacetophenone, caprylyl glycol, sodium chloride, citric acid, tetrasodium glutamate diacetate, or combinations thereof,
all amounts by weight, based on the total weight of the cosmetic cleansing composition,
wherein the cosmetic cleansing composition excludes PEG/POE surfactants, and wherein the cosmetic cleansing composition has a pH in the range from about 4.0 to about 8.0.

17. The cosmetic cleansing composition according to claim 16, wherein the at least one cationic polymer is selected from (i) chitosan present in the cosmetic cleansing composition in a range from about 0.05% to about 0.15%, (ii) polylysine present in the cosmetic cleansing composition in a range from about 0.05% to about 0.15%, (iii) guar hydroxypropyltrimonium chloride present in the cosmetic cleansing composition in a range from about 0.05% to about 0.15%, or combinations thereof, all amounts by weight, based on the total weight of the cosmetic cleansing composition.

18. A cosmetic cleansing composition comprising:
a monophasic micellar water comprising:
(a) at least one amphoteric surfactant present in an amount that is greater than about 0.05%, by weight, based on the weight of the composition;
(b) at least one cationic polymer comprising chitosan, polylysine, guar hydroxypropyltrimonium chloride, or combinations thereof, the at least one cationic polymer present in an amount that is greater than about 0.01%, by weight, based on the weight of the composition; and
(c) an aqueous carrier comprising at least water,
all amounts by weight, based on the total weight of the cosmetic cleansing composition,
wherein the cosmetic cleansing composition is essentially free of PEG/POE surfactants, and wherein the cosmetic cleansing composition has a pH of about 5, and
wherein the cosmetic cleansing composition when applied to a keratinous tissue demonstrates cleansing of longwear foundation and lipstick.

19. The cosmetic cleansing composition comprising according to claim 18, wherein the at least one amphoteric surfactant comprises (i) cocamidopropyl hydroxysultaine present in the cosmetic cleansing composition in a range from about 0.05% to about 10%, (ii) coco-betaine present in the cosmetic cleansing composition in a range from about 0.05% to about 10%, or combinations thereof, and wherein the cationic polymer is chitosan present in a range from about 0.01% to about 5%, the cosmetic cleansing composition comprising one or more co-emulsifier, and one or a combination of water-based solvents.

20. The cosmetic cleansing composition comprising according to claim 19, wherein the one or more co-emulsifier is selected from polyglyceryl-4 caprate, glyceryl caprylate, or a combination thereof present in the cosmetic cleansing composition in a range from about 0.05% to about 10%, and wherein (a) the at least one cationic polymer is selected from chitosan, guar or a combination thereof, the cosmetic cleansing composition including a blend of water soluble solvents comprising glycerin present at about 1%, and pentylene glycol present at about 3%, or (b) the at least one cationic polymer is polylysine, the cosmetic cleansing composition including a blend of solvents selected from the group consisting of (i) glycerin present at about 1%, pentylene glycol present at about 1.5% and caprylyl glycol present at about 0.5%, (ii) glycerin present at about 1%, butylene glycol present at about 3% and caprylyl glycol present at about 0.5%, (iii) glycerin present at about 1%, triethyl citrate present at about 2.5% and caprylyl glycol present at about 0.5%, (iv) glycerin present at about 1%, triethyl citrate present at about 3% and propylene glycol present at about 3%, and (v) a combination thereof.
